(19) United States
(12) Patent Application Publication
(10) Pub. No.: US 2012/0059281 A1
(43) Pub. Date: Mar. 8, 2012

(54) Intrinsic Hand Strength Measurement Device

(76) Inventors: Caterina Cleopatra Kaffes, Houston, TX (US); Jennifer Diane Cieluch, Houston, TX (US); Matthew Douglas Miller, Spokane, WA (US); Shuai Xu, Diamond Bar, CA (US); Neel Shah, Rolling Hills Estates, CA (US)

(21) Appl. No.: 13/240,878

(22) Filed: Sep. 22, 2011

Related U.S. Application Data
(63) Continuation-in-part of application No. PCT/US10/28837, filed on Mar. 26, 2010.
(60) Provisional application No. 61/164,271, filed on Mar. 27, 2009.

(51) Int. Cl. A61B 5/103

(52) U.S. Cl. 600/587

(57) ABSTRACT

A device for measuring intrinsic hand muscle strength comprising an adjustable restraint adapted to restrain at least a portion of a hand, a force transfer member adapted to fit on or around one or more digits of the hand, a force sensor connected to the force transfer member, and a processing unit connected to the force sensor is provided. Methods of measuring intrinsic hand strength are also provided.

### CROSS REFERENCE TO RELATED APPLICATIONS

This application is a continuation-in-part of International Application No. PCT/US10/28837, filed Mar. 26, 2010, which claims the benefit of U.S. Provisional Application No. 61/164,271, filed Mar. 27, 2009, the entire disclosures of which are hereby incorporated by reference.

### BACKGROUND

Intrinsic hand muscles are important for dexterity and precision movements, accordingly, their deterioration leads to a profound functional loss of the hand. Intrinsic hand muscles are located within the hand itself and account for planar lateral movement of the fingers and abduction, adduction, and flexion of the thumb. This differs from the extrinsic muscles, which are measured through pinch-grip motion. Hand trauma, rheumatoid arthritis, congenital hand defects and a host of serious pathologies, such as neurological diseases, correlate to a decrease in intrinsic hand muscle strength. Beyond blunt trauma, the assessment of Intrinsic Hand Muscle Strength (IHMS) is necessary for diseases as disparate as carpal tunnel syndrome, diabetes and median/ulnar nerve injuries. Thus, the ability to accurately quantify IHMS could aid in medical decision making, allowing for an accurate assessment of a patient's progress after rehabilitation.

Current clinical tests for measuring hand function fall into two primary methods. First, there are dexterity tests that describe hand function; however, these tests are only capable of indirectly describing intrinsic hand muscle strength (Fleishman and Hempel 2006). The second and more standardized clinical test for evaluating hand muscle strength is the Manual Muscle Test (MMT). The MMT involves ranking the patient's intrinsic muscle strength on the Medical Research Council Scale from 0 to 5, where 0 represents no contractile activity and 5 represents a full active range of motion (Shreuders et al., 2006). Although it is relatively easy to implement, the MMT lacks objectivity and sensitivity to subtle changes in hand strength. For instance, a noticeable loss of intrinsic hand muscle strength and precision from a patient's perspective would receive a similar score to no loss at all on the MMT because a relatively low amount of strength is needed to move a finger against gravity. The 5 point scale simply cannot fully capture the entire clinical spectrum. In addition, the scale also provides a dichotomy in the size between scale grades, where the difference between the grades can be different between different observers. This leads to difficult patient to patient comparisons between different clinicians.

Due to the shortcomings of clinical tests, several devices exist to measure the strength of intrinsic hand muscles more accurately and objectively. Most of the devices encountered in the literature center on the dynamometer, which is a device that is capable of measuring the force exerted by an element. Through the use of strain gauges, the device measures a change in resistance that can be converted into a force measure. Many of the dynamometers currently in use are capable of producing a wide range of available data, such as peak force, power, and endurance (An 1980). Three major devices made available in recent years include the Rotterdam Intrinsic Hand Myometer (RIHM), the Intrins-O-Meter and the device by Pataky et al.

The RIHM centers on a dyanometer for force measurements. In addition, the RIHM utilizes break tests. The patient pulls against the device, which is held stationary by the observer, until they can no longer sustain it at which point the test stops. The patient's peak force is recorded. The smallest detectable difference is about 25% of the mean of all recorded forces, which indicates relatively low sensitivity (Shreuders et al., 2004). In addition, intensive observer involvement leads to high inter-observer differences ranging from 37-52% (Shreuders et al., 2000). Finally, the device requires the measurement of one finger at a time. This leads to a longer testing scenario for the clinician and patient. Although the device is handheld, its method of acquiring data one finger at a time makes the test time consuming.

The Intrins-O-Meter operates through a dyanometer as well. Briefly, the device is placed against a patient's isolated finger. The patient is asked to either abduct or adduct a finger against the device in order to record a peak force. With the Intrins-O-Meter, the heavy clinician involvement results in similar interobserver variations as those observed with the RIHM (Shreuders et al., 2000). Due to the bulkiness of the device, measurements of internal finger adduction and abduction is substantially more difficult. The device is far easier to use when only investigating abduction of the outer fingers which minimizes interference and variability. As a sensor that reports deflection based on compression, the angle of force application greatly affects the ultimate results.

The Pataky device operates through multiple force transducers to measure both single and multifinger intrinsic muscle strength. The transducers send signals to a custom built software program. The program converts the signal strength to a calibrated force measurement. The authors suggest that intrinsic hand muscle strength be established through the maximal force of a single-finger task. In addition, the device allows for a measurement of the synergistic phenomena of “deficit” and “enslaving”. Deficit refers to a decrease in single finger forces in multifinger tasks while enslaving relates to involuntary finger forces in single finger tasks (Pataky et al. 2006). Although the Pataky device eliminates nearly all observer interference, new issues arise regarding its applicability to pediatric cases. A rigid plate design requires a patient to be able to fit their hands snugly in a flat position on the device. This means the device will be far less accommodating for patients with small hands or abnormal morphologies. The slots are approximately 2 cm across and are not adjustable. A profound difference exists between an adult hand's size which averages 180 mm long by 79 mm wide and that of a 4 or 5 year old child with an averages of 119 mm long by 53 mm wide (Snyder 1975; Agnohotri 2006). Another immediate drawback is that the strength of the thumb cannot be evaluated. Even though the Pataky device improves upon accuracy and consistency, the rigid design prevents its use as a clinical testing device for young children and/or those with abnormal hand morphologies.

### SUMMARY

The present disclosure generally relates to measuring muscle strength. More particularly, the present disclosure relates to devices and methods for measuring intrinsic hand muscle strength.

In one embodiment, the present disclosure provides a device for measuring intrinsic hand muscle strength comprising an adjustable restraint adapted to restrain at least a portion of a hand, a force transfer member adapted to fit on or around one or more digits of the hand, a force sensor connected to the force transfer member, and a processing unit connected to the force sensor. In another embodiment, the present disclosure provides methods of measuring intrinsic hand strength using a device of the present disclosure.

The features and advantages of the present invention will be readily apparent to those skilled in the art upon a reading of the description of the embodiments that follows.

### DRAWINGS

The patent or application file contains at least one drawing executed in color. Copies of this patent or patent application publication with color drawing(s) will be provided by the Office upon request and payment of the necessary fee.

Some specific example embodiments of the disclosure may be understood by referring, in part, to the following description and the accompanying drawings.

FIG. 1 is a schematic representation of an intrinsic hand strength measurement device of the present disclosure, according to one embodiment.

FIG. 2 is a schematic representation of certain components of an intrinsic hand strength measurement device of the present disclosure, according to one embodiment.

FIG. 3 are graphs showing dominant and nondominant hands combined for boys for the 5th, 25th, 50th, 75th, and 95th percentiles for (A) small finger abduction, (B) index finger abduction, (C) thumb palmar abduction, and (D) thumb opposition.

FIG. 4 are Bland-Altman plots for inter-rater reliability. The distribution along the zero line is random. The x axis is the mean of both measures of muscle strength (kgf) for (A) small finger abduction, (B) index finger abduction, (C) thumb palmar abduction, and (D) thumb opposition.

FIG. 5 are Bland-Altman plots for intra-rater reliability. The distribution along the zero line is random. The x axis is the mean of both measures of muscle strength (kgf) for (A) small finger abduction, (B) index finger abduction, (C) thumb palmar abduction, and (D) thumb opposition.

FIG. 6 are Bland-Altman plots for intra-rater reliability. The distribution along the zero line is random. The x axis is the mean of both measures of muscle strength (kgf) for (A) small finger abduction, (B) index finger abduction, (C) thumb palmar abduction, and (D) thumb opposition.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to measuring muscle strength. More particularly, the present disclosure relates to devices and methods for measuring intrinsic hand muscle strength.

In general, the present disclosure provides, according to certain embodiments, an Intrinsic Hand Strength Measurement (IHSM) device comprising an adjustable restraint, a force transfer member, a force sensor, and a processing unit. In some embodiments, an IHSM device of the present disclosure may further comprise a display panel. The overall purpose of an IHSM device of the present disclosure is to allow clinicians and researchers to quantify the intrinsic hand muscle strength of a subject by recording various strength measurements, such as peak force. endurance, etc. Accordingly, in one embodiment, an IHSM device may be used to measure intrinsic hand strength by restraining at least a portion of the subject's hand using the adjustable restraint, allowing the subject to generate force on a force transfer member connected to a force sensor using a portion of the hand that is not restrained, and measuring the force exerted on the force sensor using a processing unit to determine the subject's intrinsic hand strength. In some embodiments, these measurements are then displayed via a display panel.

One of the many potential advantages of an IHSM device of the present disclosure is that it is able to restrict nonessential hand motion, accommodate varying hand morphologies, and accurately display peak force. In addition, an IHSM device of the present disclosure differs from currently available devices in that an IHSM device allows a subject to generate force by pulling on a force transfer member, as opposed to a clinician generating force while the subject resists. Similarly, one of the major pitfalls of current commercial devices is a lack of repeatability for subjects due to a lack of restraint. The IHSM device of the present disclosure is well-suited to restrain even the most abnormal of morphologies due to its adaptability. Furthermore, as a result of this adaptability, an IHSM device of the present disclosure may be utilized with pediatric patients.

As mentioned above, an IHSM device of the present disclosure comprises an adjustable restraint. An adjustable restraint suitable for use in the present disclosure may include any structure that is capable of immobilizing a subject's hand except for that portion of the hand being tested (e.g., one or more fingers or thumb). In one embodiment, an adjustable restraint comprises a base and a plurality of adjustable restraint elements that are used to restrain a subject's hand from motion during the progression of a strength test. During testing, in one embodiment, a subject's hand is positioned onto the base and one or more adjustable restraint elements are positioned around the base of the hand and/or fingers so as to isolate extraneous hand movement. A base suitable for use in the present disclosure may be of any shape or size. Additionally, the base may be constructed from a variety of materials, including, but not limited to plastic, metal, wood, or any other sturdy material, etc. In one particular embodiment, the base may be a rectangular plastic board measuring approximately 24×24×¼ inches in width, length and thickness. Adjustable restraint elements may also be of any varying size or shape and constructed from a variety of materials, including, but not limited to plastic, metal, wood, etc. In some embodiments, adjustable restraint elements may be constructed of one material and coated in another, such as rubber.

In a preferred embodiment, the base of the adjustable restraint system comprises a plurality of holes into which adjustable restraint elements are adapted to be removably inserted into the holes. In these embodiments, the holes are generally configured in a shape and size within the base so that a portion of an adjustable restraint element fits snugly inside the hole into which it is placed. Accordingly, the size, shape and depth of the holes generally correspond to the size, shape and length of the adjustable restraint elements. In one embodiment, holes within the base may be spaced in a staggered pattern approximately 0.5 inches apart across the length and width of the base.

In addition to an adjustable restraint, an IHSM device of the present disclosure further comprises a force transfer member adapted to fit on or around one or more digits if the hand, a force sensor connected to the force transfer member, electronic circuitry connected to the force sensor, a power source connected to the electronic circuitry and force sensor, and a processing unit. During testing, while at least a portion of the subject's hand is restrained, a force transfer member is placed on or around one or more digits of the hand and the subject pulls the force transfer member. The force transfer member is connected to a force sensor that produces an electrical signal based on the force generated that is measured, amplified and converted to a digital signal and then computed to a corresponding force using a processing unit. In some embodiments, this corresponding force may then be displayed on a display panel.

Force transfer members suitable for use in the present disclosure may include anything that is capable of transferring the force generated by a subject to a force sensor. In one embodiment, a force transfer member is an adjustable loop, such as a velcro or nylon strap, that is connected to a force sensor. A force transfer member may be connected to a force sensor in any suitable manner. In one embodiment, a force transfer member is connected to a force sensor via an eye bolt.

Force sensors suitable for use in the present disclosure may include any device that is capable of converting a force into an electrical signal. In one embodiment, a suitable force sensor is a load cell comprising one or more strain gauges. In one particular embodiment, a suitable force sensor is an analog load cell comprising four strain gauges in a Wheatstone bridge configuration. In some embodiments, a force sensor suitable for use in the present disclosure is an analog load cell capable of sensing up to 10 lbs of force (0.25% resolution). Examples of suitable force sensors include, but are not limited to, the OMEGA® LCL Series thin-beam load cell and the Transducer Technique's low capacity single point load cell.

Electronic circuitry suitable for use in the present disclosure may be any circuitry that is capable of amplifying and/or filtering an electrical signal produced by a force sensor so that the signal may be processed in a processing unit. In a preferred embodiment, the circuitry is in the form of a simple Wheatstone bridge circuit with appropriate amplification and offset controls. Additionally, in some embodiments, a power source suitable for the force sensor, its associated circuitry, and/or the processing unit may be obtained from a battery.

A processing unit suitable for use in the present disclosure may comprise any data processor suitable for receiving data from a force sensor and computing a resulting force measurement. During testing, when a subject pulls the force transfer member connected to a force sensor, the force sensor produces an electrical signal that is directed to the electronic circuitry, which then amplifies and filters the signal, and the processing unit then converts the electrical signal to a force value based on the conversion factor from a calibration curve. One example of a processing unit suitable for use in the present disclosure is a microcontroller. In some embodiments, the microcontroller utilizes a software program that converts the voltage from the force sensor to force based on a calibration curve and then calculates peak force. One example of such a software program is adapted C software from LabVIEW. One example of a suitable microcontroller is an Arduino Duemilanove microcontroller. In another embodiment, a processing unit may be a computer, such as a personal digital assistant (PDA) which contains similar software.

While taking intrinsic hand strength measurements using an IHSM device of the present disclosure, it is generally desirable that the force sensor is secured so that it is stationary during testing. In one embodiment, the force sensor may be enclosed in a force sensor enclosure that is secured to the adjustable restraint. A force sensor or force sensor enclosure may be secured to the adjustable restraint in any suitable manner, including a vertically adjustable bolt or rod. In some embodiments, the force sensor or force sensor enclosure may be secured in such a manner so that the height and rotation of the force sensor or force sensor enclosure may be adjusted. As will be recognized by one of ordinary skill in the art with the benefit of this disclosure, the force sensor or force sensor enclosure may be secured in any location, including on a base of the adjustable restraint, so long as the subject is able to pull the force transfer member while the desired portion of the subject's hand is restrained. In those embodiments where a force sensor is located inside a force sensor enclosure, the force sensor enclosure may also comprise other elements such as a power source, electronic circuitry, a processing unit, etc.

In some embodiments, an IHSM device of the present disclosure further comprises a display panel connected to the processing unit. In one embodiment, the display panel may be a touch screen display, such as the TouchShield screen available from Liquidware.com. In some embodiments, a display panel may be a component of the processing unit, such as a display screen on a computer or PDA. Furthermore, in those embodiments where a force sensor enclosure is utilized, the display panel may also be a component of the force sensor enclosure.

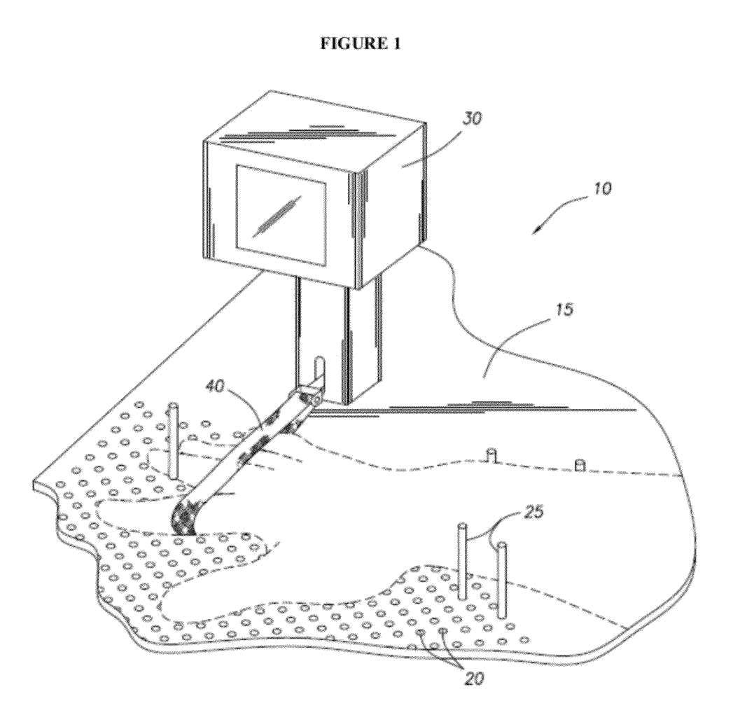

Referring now to FIG. 1, a schematic representation of an IHSM device according to one embodiment of the present disclosure is depicted. An adjustable restraint 10 is shown comprising a base 15, holes 20 and adjustable restraint elements 25, which are used to restrain at least a portion of the subject's hand from motion during the progression of the strength test. During testing, a subject's hand is positioned onto base 15 and one or more adjustable restraint elements 25 are at least partially inserted into one or more holes 20 so as to isolate extraneous hand movement. While adjustable restraint elements 25 are depicted as cylindrical in FIG. 1, they are not limited to such a configuration and may be of any varying size or shape. A person of ordinary skill in the art with the benefit of this disclosure will be able to determine the appropriate placement of one or more adjustable restraint elements 25 based on the morphology and size of the hand being tested, as well as the desired intrinsic hand muscle to be tested. As will be recognized by a person of ordinary skill in the art with the benefit of this disclosure, the hand may be positioned on base 15 with the palm facing up (e.g. to isolate thumb movements) or the palm facing down.

In the embodiment depicted in FIG. 1, once at least a portion of a subject's hand has been restrained on base 15, the subject will pull against force transfer member 40 (depicted as an adjustable loop) placed around the subject's finger(s). Force transfer member 40 is connected to a force sensor contained within force sensor enclosure 30 so that when the subject pulls on force transfer member 40, the force sensor produces a signal which is amplified and processed thereby calculating a measurement of a subject's intrinsic hand strength.

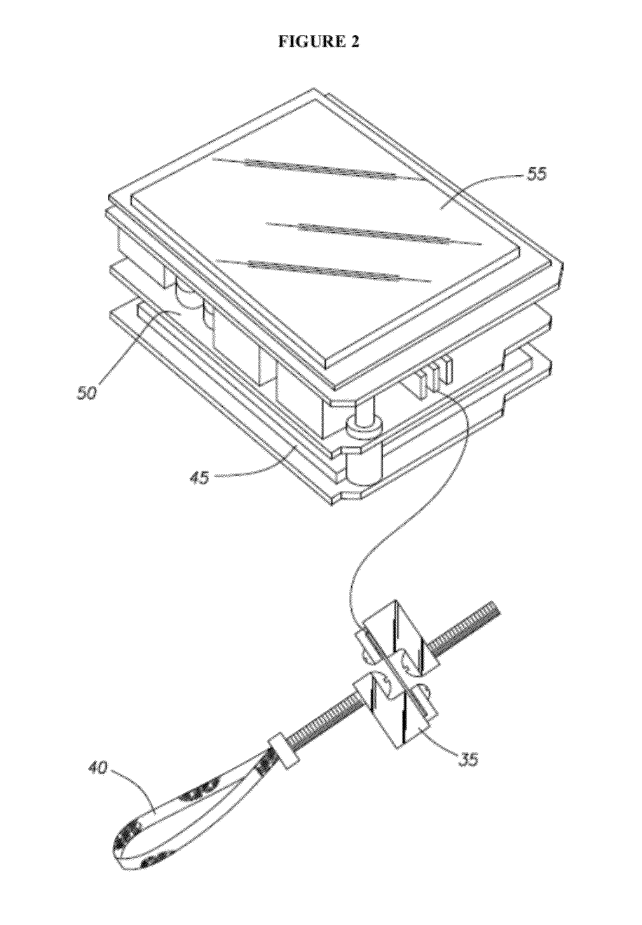

In the embodiment depicted in FIG. 1, the IHSM device comprises a force sensor enclosure 30. Referring now to FIG. 2, in one embodiment, force sensor enclosure 30 may comprise force sensor 35, which is connected to force transfer member 40 (depicted as an adjustable loop), a power source 45 which is connected to force sensor 35, and a processing unit 50 (depicted as a microcontroller). In the embodiment depicted in FIGS. 1 and 2, force sensor enclosure 30 also comprises a display panel 55. In general, force transfer member 40 is connected to force sensor 35 so that when the subject pulls on force transfer member 40, force sensor 35 produces a signal which is measured and converted to a digital signal by processing unit 50. Processing unit 50 utilizes a software program that samples voltage from the force sensor, converts the voltage to force based on a calibration curve and then calculates peak force. The results may then be displayed on display panel 55.

### EXAMPLES

Device Construction

Peg Restrained Intrinsic Muscle Evaluator (PRIME) generally consists of 4 components: an adjustable restraint in the form of a pegboard, a force transducer (e.g., force sensor), a force transfer member in the form of a hook and loop strap, and a processing unit in the form of a handheld personal digital assistant (PDA). The pegboard, with coated pegs, restrains hand and wrist movement and reduces the contribution of the extrinsic muscles. Standardized peg placement isolates individual intrinsic muscles or functional muscle groups for highly specific testing. Holes are drilled in the custom acrylic pegboard in a staggered fashion. The force transducer enclosure (FTE) is a self-contained module. Within the FTE is a load cell (LCL010; Omega Engineering, Inc., Stamford, Conn.) rated to 4.5 kg with a resolution of 0.25% o, amplification circuitry, and a battery. The interface between the FTE and the pegboard base is composed of a custom sleeve that allows for free rotation and height adjustment about the vertical axis via an adjustable lock collar. A 10-cm hook and loop strap is used to interface the force sensor with the patient's individual finger. A PDA (HP iPAQ hx2000; Hewlett-Packard Co., Palo Alto, Calif.) displays peak force through a custom program developed with LabVIEW 8.6 software (National Instrument's, Austin, Tex.). Within the PDA is a CF-6004 CompactFlash Data Acquisition Unit (National Instruments, Austin, Tex.) configured to sense analog voltage signals directly from the output of the FTE. The CF-6004 is placed in the CompactFlash slot of a PDA, and the analog signal is transmitted through a D-15 connector. The custom user interface calculates peak force from the continuously sampled data, displays the value on the PDA, and produces an exportable text (.txt) file with patient identifiers for further analysis. A zeroing function is built in to negate ambient noise from various testing locations. Calibration was done from 0 to 5 kg in 100-gram intervals. The resulting fit was highly linear (r2=0.99).

Study Participants

With institutional review board approval and written informed consent from parents, children in local summer day camps were recruited to participate in the study. Participating parents filled out a brief medical questionnaire to screen for existing neurological or musculoskeletal disorders or prior upper extremity trauma. A total of 119 children (68 boys and 51 girls), ranging from 4 to 16 years of age, participated in the study (Table 1). Of the 119 children, 107 were right-handed. For inter-rater and intra-rater measurements, a subset of 30 children (7-16 y old; 15 boys, 15 girls) from the total group was retested.

TABLE 1
Number of Participants Divided by Age Group and Gender

| Age Group (y) | Boys | Girls | Total |
|---|---|---|---|
| 4-6 | 18 | 14 | 32 |
| 7-9 | 29 | 18 | 47 |
| 10-12 | 17 | 11 | 28 |
| 13-16 | 4 | 8 | 12 |
| Total | 68 | 51 | 119 |

Testing Procedure

Participants were seated comfortably at a height-adjusted table. Hand dominance was established by asking the subject to draw a circle with a pen placed in the center of the table. The hand that the subject naturally used without prompting was recorded as the dominant hand. Participants placed their hand on the center of the pegboard, and the hook and loop strap was placed around the finger of interest and adjusted to eliminate slack. The orientation of the load cell was adjusted to be perpendicular to the force being measured. The subjects were instructed to “pull as hard as possible.” This is an isometric force from a neutral position. Peak force was automatically calculated and stored. Four specific movements were tested with the device: small finger abduction (abductor digiti minimi), index finger abduction (first dorsal interosseous), thumb palmar abduction (abductor pollicis brevis, flexor pollicis brevis), and thumb opposition (opponens pollicis, flexor pollicis brevis, adductor pollicis).

To test small finger abduction, the subject's hand is placed on the peg board with the forearm pronated. All fingers are placed in the loop, with the small finger farthest away from the load sensor. The loop is placed at the small finger proximal interphalangeal joint. Five restraining pegs are positioned: 1 on the ulnar side of the ring finger distal interphalangeal joint; 1 on either side of the wrist, 1 cm proximal to the radial and ulnar styloid; and 1 on either side of the midforearm at the most proximal peg holes available. The pegs used to restrain the forearm are positioned snugly enough to prevent motion of the forearm during testing but not to cause discomfort. Bony prominences are avoided for comfort. The subject is instructed to pull with his or her small finger against the loop, away from the load sensor. The small finger is not allowed to extend during abduction testing.

To test index finger abduction, the subject's hand is placed on the peg board with the forearm pronated. All fingers are placed in the loop, with the index finger farthest away from the load sensor. The loop is placed at the index finger proximal interphalangeal joint. Five restraining pegs are positioned: 1 on the radial side of the long finger distal interphalangeal joint, and the other 4 as previously described for small finger abduction. The subject is instructed to pull with his or her index finger against the loop, away from the load sensor. The index finger is not allowed to extend during abduction testing.

To test thumb palmar abduction, the subject's hand is placed on the peg board on its ulnar border with the forearm in neutral rotation. The back of the hand faces the load sensor. The enclosure itself is moved up the vertical axis so that the loop is level with the thumb. The loop is placed around the thumb metacarpophalangeal joint. A restraining peg is placed in line with the distal palmar crease, and the subject can flex his or her fingers around this peg. The wrist is positioned in 10° of extension, and 4 restraining pegs are placed along the volar and dorsal forearm, 2 distally and 2 at the most proximal peg holes available. The forearm pegs are positioned snugly enough to prevent motion of the forearm during testing but not so tight as to cause discomfort. The subject is instructed to abduct his or her thumb in a palmar direction.

To test thumb opposition, the subject's hand is rested on the peg board with the forearm in supination. The radial border of the hand faces the load sensor. The device is adjusted on the vertical axis so that the loop is level with the thumb. The loop is placed around the thumb metacarpophalangeal joint, taking care that it does not slip distally during testing. Four restraining pegs are placed along the volar and dorsal forearm, 2 distally and 2 at the most proximal peg holes available. The forearm pegs are positioned snugly enough to prevent motion of the forearm during testing but not so tight as to cause discomfort. The subject is instructed to touch the tip of his or her thumb to the tip of the small finger. The clinician restrains the 4 fingers, allowing only thumb motion.

Participants performed 3 trials for each muscle, with all results reported as mean force (kg). To determine inter-rater reliability, 2 different examiners sequentially tested each subject in that subset on the same day, with a 15- to 20-minute rest period between testers. For intra-rater reliability, the same examiner re-evaluated each subject in that subset within an average of 2 days (range, 1-7 d).

Statistical Methods

Normative data is presented as means with standard deviations. We calculated intraclass correlation coefficient (ICC), standard error of measurement (SEM) and smallest detectable difference (SDD). ICC is the ratio between patient variance and total variance. The standard error of measurement (SEM) is calculated by taking the SD*(1−ICC). The SDD is smallest difference that represents meaningful clinical change. SDD is calculated as 1.96×√2×SEM. Visualization was done with Bland-Altman plots. For normative data, the measurements of PRIME are presented using a crossover design. Such a design allows for greater precision when less variability is expected within subjects than between subjects. For each of the four muscles tested, three PRIME measurements were taken per subject. Therefore, analysis precludes a technique that optimizes information from this specific crossover design. For this reason, we used a Repeated Measures Analysis of Variance with a random subject effect. This method estimates and compares treatment effects while at the same time accounting for both between and within subject correlation. In each model, we show 5%, 25%, 50%, 75% and 95% percentiles. Quantile regression provides conditional predictions of designated quantiles of the population with respect to PRIME in order to examine the differential effect of predictors. For example, the 0.05 or 5% quantile of observed PRIME may behave differently from the median or 50% quantile. We utilized quantile regression to partition the population estimates of PRIME into 100 quantiles (percentiles) and estimate the effect of the predictors on specifically the 5, 25, 50, 75 and 95 percentiles. All calculations were completed using SPSS 16.0 and SAS 9.0 software.

Results

Normative Data. Statistical analysis demonstrated that gender and age were significant determinants of intrinsic hand strength for all four muscles tested. Results for 4-12 year old girls and boys are summarized in Tables 3 and 4. All strength measures increased with age (p<0.0001). Boys demonstrated higher strength than girls (p=0.006) for all muscle groups in all age groups. The greatest difference between genders was seen in the 10-12 year old group. Of note, the 13-16 year old group was not analyzed for gender differences due to insufficient power. Mean ADM and 1^{st} DI strength was significantly lower than APB and Opponens for both genders.

TABLE 2
Intrinsic Hand Muscle Strength for Girls

| Age | | | | | 95% o | | | | | 95% o |
|---|---|---|---|---|---|---|---|---|---|---|
| Group | n* | Mean | SD | Range | Limits | n* | Mean | SD | Range | Limits |
| | Hypothenar (kgf) | | | | | First Dorsal Interosseous (kgf) | | | | |
| 4-6 | 28 | 0.3 | 0.2 | 0.1-0.6 | 0.2-0.4 | 28 | 0.7 | 0.5 | 0.2-1.5 | 0.5-1.0 |
| 7-9 | 36 | 0.4 | 0.2 | 0.1-0.9 | 0.3-0.6 | 36 | 0.9 | 0.4 | 0.3-1.5 | 0.7-1.1 |
| 10-12 | 22 | 0.5 | 0.3 | 0.1-1.1 | 0.4-0.7 | 20 | 1.2 | 0.7 | 0.3-2.1 | 0.7-1.7 |
| | Abductor Pollicis Brevis (kgf) | | | | | Thumb Opposition (kgf) | | | | |
| 4-6 | 26 | 1.0 | 0.5 | 0.2-2.0 | 0.6-1.3 | 24 | 0.9 | 0.4 | 0.2-1.5 | 0.6 |
| 7-9 | 36 | 1.3 | 0.5 | 0.5-2.6 | 1.0-1.5 | 36 | 1.1 | 0.7 | 0.3-2.6 | 0.8 |
| 10-12 | 20 | 1.5 | 0.6 | 041-2.2 | 1.0-1.9 | 20 | 1.4 | 0.7 | 0.4-2.3 | 0.9 |

*n, number of hands tested.

TABLE 3
Intrinsic Hand Muscle Strength for Boys

| Age | | | | | 95% o | | | | | 95% o |
|---|---|---|---|---|---|---|---|---|---|---|
| Group | n* | Mean | SD | Range | Limits | n* | Mean | SD | Range | Limits |
| | Hypothenar (kgf) | | | | | First Dorsal Interosseous (kgf) | | | | |
| 4-6 | 36 | 0.4 | 0.1 | 0.2-0.6 | 0.3-0.4 | 34 | 0.9 | 0.3 | 0.4-1.7 | 0.7-1.0 |
| 7-9 | 54 | 0.4 | 0.2 | 0.1-0.9 | 0.4-0.5 | 54 | 1.1 | 0.4 | 0.2-1.8 | 0.9-1.2 |
| 10-12 | 34 | 0.8 | 0.3 | 0.2-1.4 | 0.7-1.0 | 34 | 1.5 | 0.4 | 0.6-2.3 | 1.3-1.7 |
| | Abductor Pollicis Brevis (kgf) | | | | | Thumb Opposition (kgf) | | | | |
| 4-6 | 30 | 1.2 | 0.5 | 0.3-2.1 | 1.0-1.5 | 32 | 1.0 | 0.5 | 0.2-2.0 | 0.6 |
| 7-9 | 54 | 1.5 | 0.6 | 0.4-3.0 | 1.2-1.7 | 58 | 1.6 | 0.7 | 0.4-3.5 | 0.8 |
| 10-12 | 34 | 2.5 | 0.9 | 1.1-4.1 | 2.0-2.9 | 34 | 2.3 | 0.8 | 1.4-3.9 | 0.9 |

*n, number of hands tested.

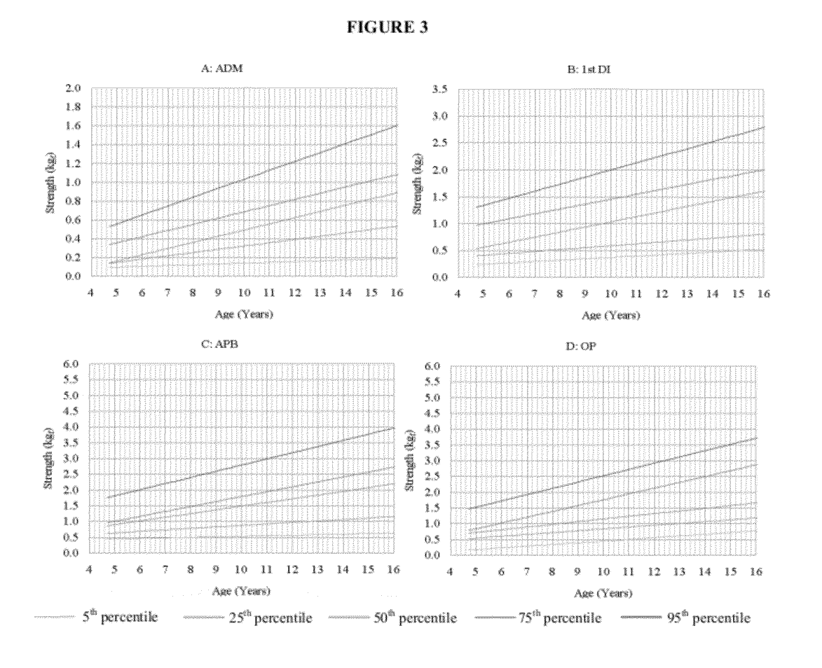

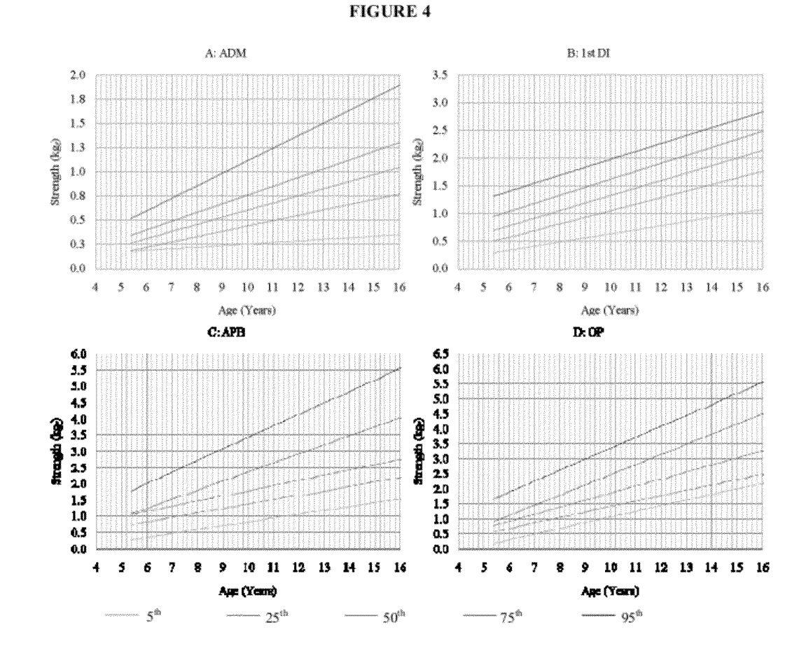

Although the dominant hand generally produced higher strength scores, the difference between dominant and non-dominant hands was not statistically significant (p=0.376). To simplify clinical interpretation, dominant and non-dominant hand intrinsic strength is combined and plotted as a function of age and gender for each muscle tested. 5%, 25%, 50%, 75% and 95% percentiles were fitted along a continuum of age to describe the progression of intrinsic hand strength in girls (FIG. 3) and boys (FIG. 4). This presentation is similar to the normative growth charts clinicians typically use to follow children's height and weight over time.

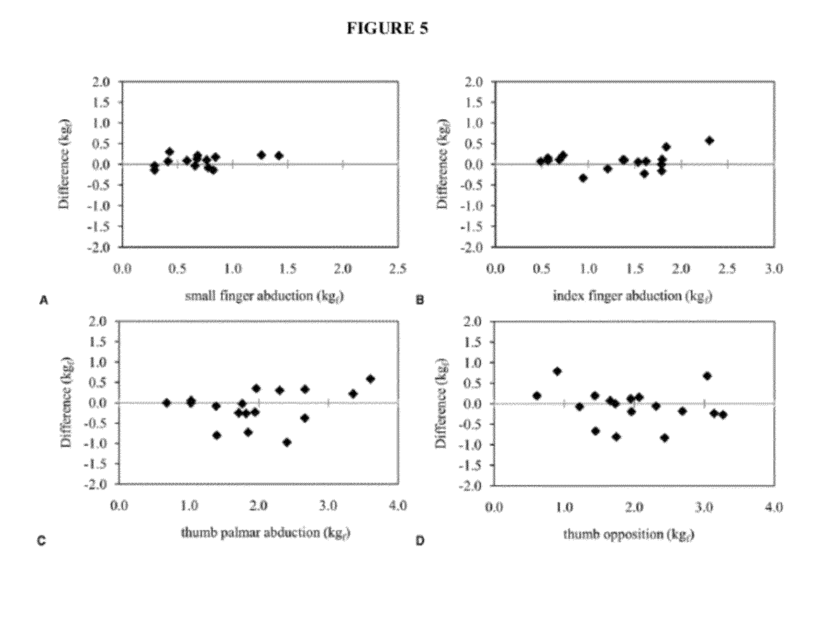

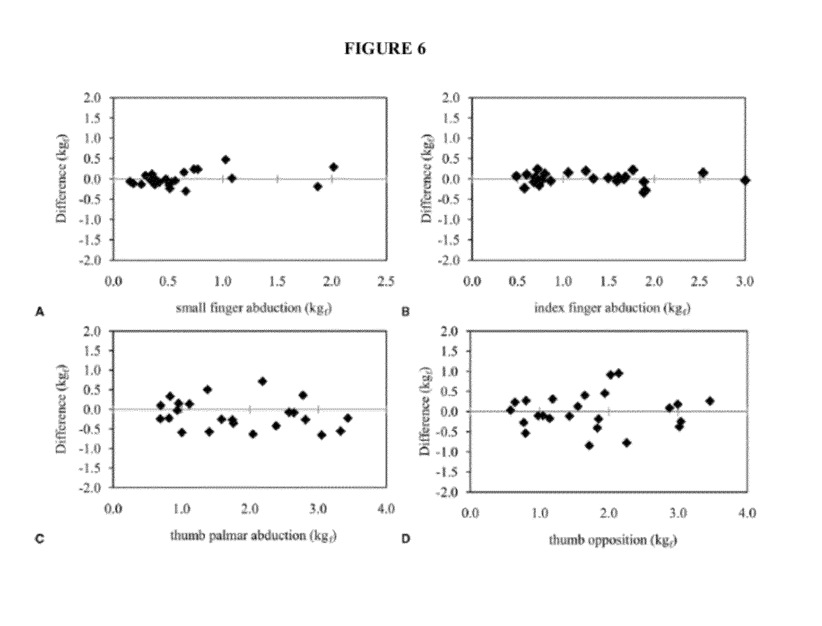

Reliability. Table 4 summarizes the ICC, SDD, and SEM results for inter- and intra-rater reliability. The ICCs ranged from 0.85 to 0.94 for inter-rater reliability and 0.88 to 0.98 for intra-rater reliability. The thumb muscles exhibited lower intra-rater and inter-rater reliability than the abductor digiti minimi and first dorsal interosseous. In turn, thumb palmar abduction and opposition show the highest SDDs (0.76 kgf to 0.85 kgf). The Bland-Altman plots for both inter-rater and intra-rater reliability indicate an even distribution along the zero line for all tests, suggesting the absence of systematic bias (FIGS. 5, 6). No substantial difference in reliability was seen between boys and girls or between dominant and nondominant hands.

TABLE 4
Inter-Rater and Intra-Rater Repeatability Results for PRIME

| | Inter-Rater | | | | Intra-Rater | | | |
|---|---|---|---|---|---|---|---|---|
| | | | SDD | | | | SDD | |
| Muscle Group | n* | ICC | (kgf) | SEM (kgf) | n* | ICC | (kgf) | SEM (kgf) |
| Hypothenar | 16 | 0.90 | 0.29 | 0.10 | 23 | 0.93 | 0.35 | 0.13 |
| First dorsal interosseous | 16 | 0.94 | 0.34 | 0.12 | 24 | 0.98 | 0.28 | 0.10 |
| Thumb Abductor pollicis | 17 | 0.85 | 0.84 | 0.30 | 23 | 0.91 | 0.76 | 0.27 |
| br4evis | | | | | | | | |
| Opposition | 17 | 0.86 | 0.83 | 0.30 | 24 | 0.88 | 0.85 | 0.31 |

*n, number of hands tested.

Reliability.

The PRIME device is a reliable tool for the quantification of intrinsic hand muscle strength in children. Age-specific and gender-specific normative values in typically developing children can serve as a future resource for clinicians treating pediatric hand or neuromuscular conditions.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

### REFERENCES

Dias, J J, Garcia-Elias M. (2006). Hand injury costs. Injury, 37, 1071-1077.
Sorock G S et al. (2001). Epidemiology of occupational acute traumatic hand injuries: a literature review. Safety Science, 38, 241-256.
Hastings H II, Simmons B P. (1984). Hand fractures in children. A statistical analysis. Clin Orthop Relat Res, 188, 120-130.
Estes J P, Bochenek C, Fassler P. (2000). Osteoarthritis of the fingers. J Hand Ther, 13, 108-23.
Ardic F, Soyupek F, Kahraman Y, Yorgancioglu R. (2003). The musculoskeletal complications seen in type II diabetics: predominance of hand involvement. Clin Rheumatol, 22, 229-33.
Lundborg G. (1993). Peripheral nerve injuries: pathophysiology and strategies for treatment. J Hand Ther 1993, 6, 179-88.
Richard A. C. Hughes. (2008). Treating nerves: a call to arms. Journal of the Peripheral Nervous System, 13, 105-111.
Kaufman K et al. (1991). Dynamic joint forces during knee isokinetic exercise. Am J Sports Med, 19, 305-16.
Chan K M, Maffulli N, Korkia P, Li C T. (1996). Principles and practice of isokinetics in sports medicine and rehabilitation. Hong Kong: Williams & Wilkins.
Kannus P. (1994). Isokinetic evaluation of muscular performance: implication for muscle testing and rehabilitation. Int J Sports Med, 15, S11-8.
Mannerfelt L. (1997). Studies on ulnar nerve compression neuropathies with a new computerized instrument—the Intrins-o-meter. Scand J Plast and Reconstr. Surg. Hand Surg, 31, 251-260.
Schreuders T A, Selles R W, Roebroeck M E, Stam H J. (2006). Strength measurements of the intrinsic hand muscles: A Review of the Development and Evaluation of the Rotterdam Intrinsic Hand Myometer. J Hand Ther, 19, 393-402.
Pataky T C, Savescu A V, Latash M L, Zatsiorsky V M. (2007). A device for testing the intrinsic muscles of the hand. J Hand Ther, 20, 345-350.
Schreuders T A, Roebroeck M, van der Kar T J, Soeters J N, Hovius S E, Stam H J. (2000). Strength of the intrinsic muscles of the hand measured with a hand-held dynamometer: reliability in patients with ulnar and median nerve paralysis. J Hand Surg [Br], 25, 560-5.

What is claimed is:

1. A device for measuring intrinsic hand muscle strength comprising:
an adjustable restraint adapted to restrain at least a portion of a hand;
a force transfer member adapted to fit on or around one or more digits of the hand;
a force sensor connected to the force transfer member; and
a processing unit connected to the force sensor.

2. The device of claim 1 wherein the adjustable restraint comprises a base and one or more adjustable restraint elements.

3. The device of claim 2 wherein the base has a plurality of holes formed therein.

4. The device of claim 3 wherein the one or more adjustable restraint elements are adapted to be removably insertable into the holes of the base.

5. The device of claim 1 wherein the force sensor is an analog load sensor.

6. The device of claim 1 wherein the force transfer member is an adjustable loop.

7. The device of claim 1 wherein the processing unit is a microcontroller.

8. The device of claim 1 wherein the processing unit is a computer.

9. The device of claim 1 further comprising electronic circuitry.

10. The device of claim 1 further comprising a power source.

11. The device of claim 1 further comprising a display panel.

12. The device of claim 11 wherein the display panel is a touch screen panel.

13. The device of claim 1 wherein the processing unit utilizes a software program that samples voltage from the force sensor and converts the voltage to a corresponding force measurement.

14. A method comprising:
providing an intrinsic hand strength measurement device comprising an adjustable restraint adapted to restrain at least a portion of a hand, a force transfer member adapted to fit on or around one or more digits of the hand, a force sensor connected to the force transfer member, and a processing unit connected to the force sensor;
allowing a subject to apply force to the force transfer member while at least a portion of the subject's hand is restrained by the adjustable restraint; and
measuring the force exerted using the processing unit.

15. The method of claim 14 wherein the adjustable restraint comprises a base and one or more adjustable restraint elements.

16. The method of claim 15 wherein the base has a plurality of holes formed therein.

17. The method of claim 16 wherein the one or more adjustable restraint elements are adapted to be removably insertable into the holes of the base.

18. The method of claim 14 wherein the force sensor is an analog load sensor.

19. The method of claim 14 wherein the force transfer member is an adjustable loop.

20. The method of claim 14 wherein the processing unit is a microcontroller.

21. The method of claim 14 wherein the processing unit is a computer.

22. The method of claim 14 wherein the intrinsic hand strength measurement device further comprises electronic circuitry.

23. The method of claim 14 wherein the intrinsic hand strength measurement device further comprises a power source.

24. The method of claim 14 wherein the intrinsic hand strength measurement device further comprises a display panel.

25. The method of claim 24 wherein the display panel is a touch screen panel.

26. The method of claim 14 wherein the processing unit utilizes a software program that samples voltage from the force sensor and converts the voltage to a corresponding force measurement.
